(19) United States
(12) Patent Application Publication
(10) Pub. No.: US 2011/0263925 A1
(43) Pub. Date: Oct. 27, 2011

(54) Pulsed Magnetic Therapy Device

(76) Inventor: Charles Bratton, Honolulu, HI (US)

(21) Appl. No.: 13/093,766

(22) Filed: Apr. 25, 2011

Related U.S. Application Data
(60) Provisional application No. 61/327,116, filed on Apr. 23, 2010.

(51) Int. Cl. A61N 2/04

(52) U.S. Cl. 600/14

(57) ABSTRACT

A device for treating diseases and chronic ailments of the human body by inducing powerful, short duration magnetic pulses in close proximity to a region of the body. The device comprises an electromagnetic coil or inductor energized by an electric circuit and a power source, pulsed between 1 and 25 pulses per second for therapeutic means and use as an alternative medical treatment. The device comprises an electric circuit that transmits pulsating current to an induction coil placed within a handheld stylus device for introducing a high energy, pulsed magnetic field to target locations on the body. The stylus is placed against the human body and introduces the pulsating magnetic field into and through body tissue, bone and the bloodstream. The stylus is completely noninvasive and provides a means to direct magnetic energy to a specific part of the body. The device is an advancement in the art, and one that provides increased magnetic energy and pulse frequency without the use of Xenon flash tubes that consume large quantities of power.

### CROSS REFERENCE TO RELATED APPLICATION

This application claims the benefit of U.S. Provisional Application No. 61/327,116 filed on Apr. 23, 2010, entitled “Ultimate Magnetic Pulser.”

### BACKGROUND OF THE INVENTION

1. Field of the Invention

The present invention relates to alternative medical devices and magnetic therapy. More specifically, the present invention pertains to a pulsed electromagnet device for inducing a magnetic field in proximity to a user for therapeutic purposes and improved health.

2. Description of the Prior Art

Magnetic therapy is an alternative form of medical therapy that is believed to affect blood vessels, improve healing and recovery of tissue, and reduce inflammation in the human body. While it is considered an alternative form of treatment, its use has proven very effective for many individuals suffering from chronic diseases, ailments and physical damage. Traditional magnetic therapy utilizes permanent magnets, which are placed in close relation to a patient. The magnet produces a magnetic field in close proximity to the body that penetrates bone and tissue. It is known that certain cells of the body, pathogens and other organisms have a slight diamagnetic property that may be affected by the nearby magnetic field. The goal of magnetic therapy is to introduce a magnetic field through the body to affect a change in the normal operation of these articles for the purposes of improving health, reducing disease effectiveness and to regrowing tissue in damaged areas. Increased blood flow and pain relief are also purported effects of magnetic therapy.

More recently, advances in electro-magnetic therapy have shown to be more effective than static or permanent magnet therapy. Electro-magnetic therapy, and more specifically pulsed electro-magnetic therapy (PEMFT), is a form of magnetic therapy wherein a magnetic coil is utilized to produce a fluctuating magnetic field. An electric power source and controller produces a pulsed waveform excitation into the coils, which are placed on the surface of a user's body for penetration into tissue and blood vessels. Transient electric current, controlled by an electric circuit, is supplied to the wire-wound coil that produces the electromagnetic field, producing a standing magnetic waveform with a given pulse rate and amplitude.

The effect of magnetic pulse therapy is believed to induce electric currents in the human body and in the blood stream. The primary function of the disclosed invention is to reduce inflammation and pain in areas of the body and to improve circulation of blood through tissue. The improved circulation promotes faster heating times and recovery, as well as strengthens organs and tissue to allow an overall positive health benefit from the device. The therapy works by inducing magnetic pulses onto an affected area, whereby the electric membrane potential of cells is gradually restored to a healthy level.

Alternate functions include stimulating lymph flow so that immune cells have unimpeded flow from bone marrow into the bloodstream, where they can effectively fight infection and pathogens. The device improves overall blood flow to improve immune system function and flush out viruses from peripheral veins of the lymph system, and reduces electrical attraction between viruses and other cells to keep them freely circulating in the bloodstream. The therapy may be utilized with other alternative medical protocols, such as blood electrification and methods that improve oxygen in the blood stream. Together, the therapy is believed to disable or kill viruses, bacteria and other pathogens in the body, as well as improve tissue oxygenation and blood flow for improved health, particularly for those with chronic or incurable conditions such as cancer, Epstein bar and HIV.

Early methods of electro-magnetic therapy and blood electrification included surgical implementation of such devices within a cavity of a user for a period of time. The device implanted and removed or refreshed after the given power supply had depleted. This method is invasive to the user and problematic. Introduction of a third-party object within the body of a user can lead to complications during surgery, swelling and pain, as well as the risk of infection around the imbedded object. The short lifespan of the device is also a major drawback, as it requires additional surgery to replace or remove the device after power has been depleted.

Advances in the art produced external magnetic and electro-therapy means, including the use of magnetic coils attached to handled-instruments, capable of introducing sufficient magnetic fields and fluxes throughout the body from an external source. The present invention relates to such devices, more specifically to a rapidly pulsed magnetic coil device directed at particular areas of the body using a stylus device. The pulsed magnetic field penetrates the skin and bones and induces a magnetic field within the body, which has an effective range of 9 to 12 inches. Pulses are controlled by an electric circuit, which is designed to allow the coil to pulse at up to 25 pulses per second. The voltage of the pulse is lower than currently available devices, but the frequency of pulses is increased while still providing an effective range that can penetrate through the thickness of a human body. No Xenon flash tubes are utilized, which are common components to devices in the art that consume large amounts of energy and reduce the ability to pulse the coils at a high rate. Finally, a perfect layer coil is utilized in the stylus, which provides a fast response time and maintains a pulse as it is dropped and reenergized at a fast rate.

Devices in the art have been suggested for creating a pulsed magnetic field for therapeutic purposes. The prior art devices have several known drawbacks that the present invention addresses. Most notably, the present invention provides a portable device with a body-directable stylus. The stylus comprises a handle region and a working end of embedded coil windings, which are powered by a pulsed, high output electric circuit device. Power is delivered to the coil at a given pulse rate, and the magnetic field resembles a square wave that is considerably more powerful than currently available pulsing magnetic therapy devices.

U.S. Pat. No. 6,280,376 to Halcomb describes one such device, in which an electromagnetic treatment device is disclosed. While this device introduces a magnetic field into the patient, its structure is sufficiently large and complex to prevent portability and ease of use. Embodiments of the device show its use in conjunction with a patient table or a chair structure, as opposed to a handheld device as disclosed in the instant invention.

U.S. Pat. No. 5,014,699 to Pollack describes another such device, in which a portable electromagnetic therapy method and apparatus are disclosed. This device utilizes a variety of transducers to apply a magnetic field around a bone fracture, particularly a nonunion or delayed union fracture wherein surgery may be required if considerable healing occurs to bridge the nonunion. While this device may promote bone growth, its structure is battery powered and its output power is limited as compared to the present invention. This device would be suited for long-term periods, in a cast or splint device.

U.S. Pat. No. 6,443,883 to Ostrow describes a similar device as the Pollack patent, wherein a pulsed magnetic therapy device is disclosed for the purposes of improving bone growth in a cast placed over a fracture site. Similar to the aforementioned patent, this device relies on an internal battery for a power supply, which limits its output power and magnetic field strength.

U.S. Pat. No. 5,842,966 to Markoll describes another pulsed magnetic therapy device, wherein a large annular coil device is utilized to surround an organ or limb of the human body. The coil produces a pulsed magnetic field at a rate of 1 to 30 cycles per second. While this devices is powered by an AC power source, and can produce high frequency and high energy magnetic fluxes, its use is limited due to its size and construction. The annular coil requires a body part to be placed within its center, limiting the device to treating limbs of the body. Its large size is prohibitive as well, when compared to the portability and modularity of the present invention.

U.S. Pat. No. 6,234,953 to Thomas describes an apparatus and method of treating physiological, neurological and behavior disorders by subjecting patients to a low frequency pulsed magnet therapy. This device differs from the present invention in structure and intent. The prior art patent subjects low frequency magnetic pulses to a specific tissue utilizing a plurality of intermittent waveforms. The present invention utilizes a high output, pulsed wave to treat body tissue for short periods of time, as opposed to an intermittent signal with a variety of qualities.

U.S. Pat. No. 7,175,587 to Gordon describes a pulsed electromagnetic therapy device in which a straight wire element is employed to induce a low energy square wave on portions of the body, directed to specific locations using a handheld probe or a deformable pad placed over an affected area. Peak field strength of the magnetic field is approximately 2 gauss at 1 centimeter from the straight wire element, which is considerably less than the field produced with the present invention. The present invention utilizes a magnetic field strength of approximately 55 gauss at 2 millimeters from a coil wire device. The apparatus used to induce the magnetic field also differs between the prior art patent and the present invention.

U.S. Pat. No. 5,085,626 to Frey describes a magnetic energy device for treating cells and tissues of the human body, comprising an induction coil and linear core fixed within the coil. An electric current generator is utilized to feed the coil current and induce the magnetic field, while the coil is placed on the patient with a looping strap device. The structure of the coil/core differs from the present invention, which does not utilize a core to induce a magnetic field.

U.S. Pat. No. 6,955,642 to Simon discloses a noninvasive method and apparatus involving pulsed electromagnetic fields. The magnetic fields are approximately 4-10 micro Tesla, while the frequency of pulsation is a saw tooth wave. This differs from the present invention, wherein a higher energy magnetic field is generated (550 micro Tesla) and in a waveform resembling a chopped square wave.

U.S. Pat. No. 5,527,259 to Grace describes another method and apparatus for therapeutic treatment with pulsed magnetic therapy. The frequency of the magnetic field is in the range of 0.5 Hz to 25 Hz; however the signal includes considerable higher harmonics and noise. The higher harmonics create a static waveform, as opposed to the present invention, which forms a pulsed square waveform of alternating magnetic field strength.

Finally, U.S. Pat. No. 5,030,196 to Inoue describes a pulsed magnetic treatment device comprising a coil device and handheld stylus for application to different parts of the human body. While this device provides an oscillating electromagnetic field in a handheld device, its power source is a DC battery. This limits the output power potential, reduces effectiveness as the battery is slowly drained, and finally requires periodically changing out the batteries for a fresh set. The present invention utilizes a power cord attachable to any wall outlet for drawing continuous AC power.

The devices in the art do not address the need for a handheld, portable pulsed electromagnetic therapy device. Particularly one that provides a high energy magnetic field pulse that fluctuates at a high rate for short periods of time. The present invention utilizes an electric control circuit for utilization of AC power and distributing current to a coil device positioned on the working end of a handheld stylus, which is placed on the surface of patient in an affected area requiring treatment. It substantially diverges in design elements from the prior art, consequently it is clear that there is a need in the art for an improvement to pulsed electromagnetic therapy devices for treating various ailments and inflammation. In this regard the instant invention substantially fulfills these needs.

### SUMMARY OF THE INVENTION

In view of the foregoing disadvantages inherent in the known types of magnetic pulse therapy devices now present in the prior art, the present invention provides a new magnetic pulse therapy device wherein the same can be utilized for providing convenience for the user when inducing a pulsed frequency, high energy magnetic field in close proximity to the human body for improving health, increasing immune system function and increasing blood flow.

It is therefore an object of the present invention to provide an electric circuit that provides current to an induction coil device, placed in the working end of a handheld stylus, for the purposes of inducing a pulsing magnetic field through a user's body.

Another object of the present invention is to provide an induction coil and electric circuit that accepts household AC electric, powers the induction coil magnetic field to 55 gauss pulsed at a pulsed rate up to 25 Hz in a square wave function.

Another object of the present invention is to provide a pulsed electromagnetic therapy device that delivers higher power magnetic fields than current devices, at a high pulse output frequency.

Another object of present invention is to provide a housing and controller for the induction coil that is easily manufactured and composed of readily available electronic components.

Another object of the present invention is to provide a timing circuit that will shut the device off after a period of use or after sufficient heat is developed that would impede use or introduce a fire hazard within a home.

Yet another object of the present invention is to provide a method of pulsed electromagnetic therapy wherein the user places a handheld induction coil over a specific area of the body, and introduces high energy, pulsed magnetic fields into the body to reduce inflammation, increase immune function and increase blood flow.

Finally, it is an object of the present invention to provide a new and improved pulsed electromagnetic therapy device that has all of the advantages of the prior art and none of the disadvantages.

Other objects, features and advantages of the present invention will become apparent from the following detailed description taken in conjunction with the accompanying drawings.

### BRIEF DESCRIPTIONS OF THE DRAWINGS

FIG. 1 shows a perspective view of the present invention, including the electric circuit housing, controls, and LED display and a handheld stylus.

FIG. 2 shows the preferred magnetic field waveform according to the preferred embodiment of the present invention.

FIG. 3 shows a schematic of an embodiment of the electric circuit controlling and powering the present invention.

### DETAILED DESCRIPTION OF THE INVENTION

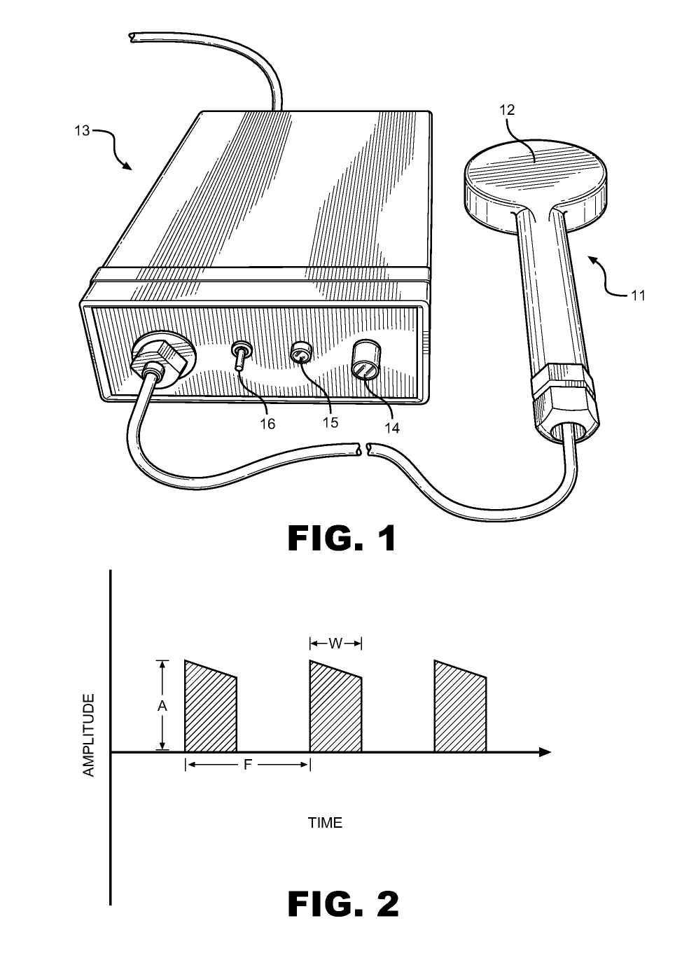

Referring now to FIG. 1, there is shown a perspective view of the preferred embodiment of the present invention. A handheld stylus 11 with a working end 12 attaches to an electrical circuit housing 13 via an elongated electric cable. The electrical cable transmits current from the housing 13 to the stylus 11 during operation. Along the outside of the housing 13 are a plurality of controls and indicators, including a frequency knob 14, an operational LED light 15 and an on/off switch 16. Along the backside of the housing is an AC connector and a slide switch for alternating between 110 and 220 volt AC input.

Imbedded within the working end of the stylus 12 is an induction coil, comprising a winding of 18 AWG, 10 mH Perfect Layer Inductor coil. Layers of the winding are wound very tightly and bounded together to keep DC resistance to a minimum. The windings are wound around an open core section, which ensures no saturation, hysteresis or any other magnetic distortions. Electrical current is supplied to the coil through the stylus 11 attachment wire, originating within the housing at the output of the electric circuit.

The controls on the front of the housing 13 provide control over the magnetic field produced from the induction coil. The frequency knob 14 controls the output frequency from the electrical current, and therefore the frequency with which the magnetic field is pulsed. The device comprises a frequency range of 1 Hz to 25 Hz. The operational light 15 is a light emitting diode that provides the user with an operational signal light, indicating the device output frequency. The diode is flashed every time the magnetic field is pulsed through the coil. Finally the on/off switch 16 provides control over operation of the device.

Referring now to FIG. 2, there is shown an example magnetic field waveform according to the preferred embodiment of the present invention. The graph shows pulses in the magnetic field in the form of a truncated square wave. Utilization of the air-core perfect layer inductance coil provides rapid response time and maintains the pulse as it is dropped and reenergized at a rapid pulse rate. A 555 timing circuit determines the output interval between each magnetic pulse, which is controllable by a knob on the device housing. The range of frequency (f) varies up to 25 Hz, which translates into a pulse every 43 milliseconds. The pulse width (w) is between 4.15 to 4.3 milliseconds in duration. In operation, the device has been measured at 55,000 mGauss using a MAGSYS HGM09 gauss meter at 2 mm from the coil face. The present invention therefore provides a very high-powered, fluctuating magnetic field with a high pulsing frequency when compared to similar devices on the market and in the art.

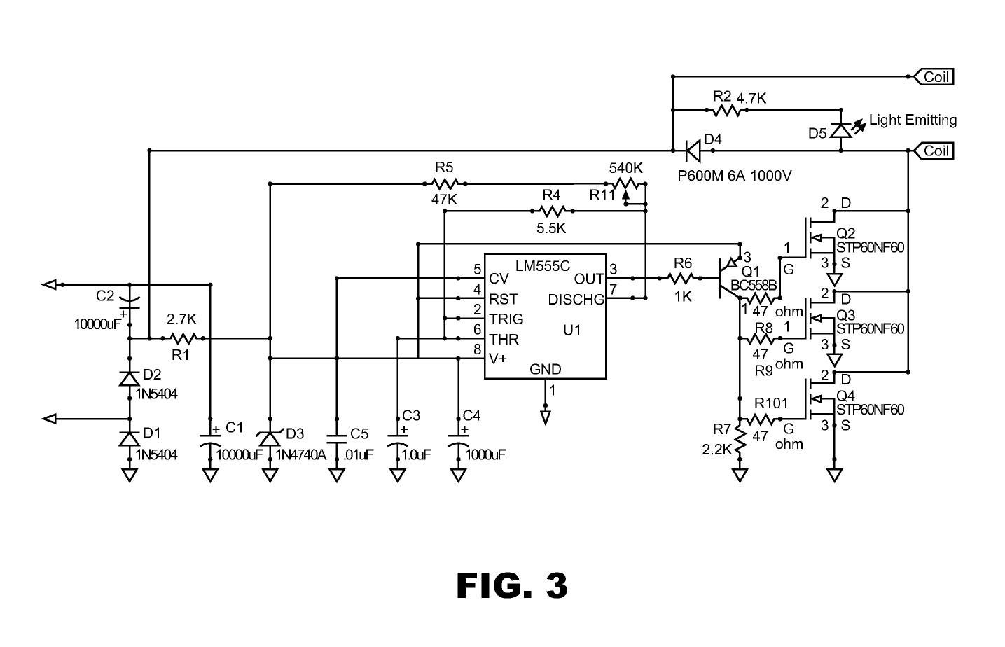

Referring now to FIG. 3, there is shown a circuit diagram schematic of a preferred embodiment of the present invention. The electric circuit functions by dropping incoming AC voltage down to between 16 to 24 volts AC. The voltage is then put through a voltage doubler converter which increases it to about 58 volts and also converts from AC to DC. Part of the DC voltage is dropped down to 9 volts to run a 555 timing circuit that determines the timing and pulse width of the signal that is sent to the coil imbedded within the stylus. The other higher voltage is switched on and off by 3 MOSFET transistors that feed the coil in the paddle.

The circuit design and operation of the device is completely solid state, including no moving parts. Commonly found on pulsing electromagnetic therapy devices are Xenon flash tubes, which require considerable amounts of power. The present invention is designed without flash tubes for maximum power delivery to the inductance coil during operation. No external batteries or power supplies are required, as the device accepts 120 volts AC or 240 volts AC with an IEC cord or power plug adapter. The primary components that provide energy storage and magnetic flux are a 20,000 uF capacitance and a 10 mH inductance coil.

Xenon flash tubes are electronic discharge devices or switches that are commonly used in electromagnetic therapy devices. They discharge a large amount of energy stored in a capacitor bank, but they consume a considerable amount of energy that is wasted as heat and light. Places an inductance coil in series with a Xenon flash tube forces part of the same discharge to go through the coil and create a magnetic pulse. The coils tend to block the flow of energy unless they energy is released very quickly. The use of such devices is eliminated from the present invention to reduce power consumption and waste.

The disclosed circuit design pulses the induction coil at a rate of 10 percent of the overall signal. The rest time between pulses is therefore 90 percent, which prevents the coil from developing a resistance to the signal. The coil is rated at a higher inductance (10 mH) than most prior art patents, which are closer to 2.5 mH. The disclosed coil therefore produces a higher energy magnetic field and pulses at a faster rate.

In use an individual targets inflamed locations on the body or strategic locations for specific conditions. The working end of the stylus is placed against the user's body around the area of interest, and the device is activated. Power is supplied via a standard electrical outlet, fed into the circuit, and modified therein. A flow of current is fed to the inductance coil at the working end of the stylus, and pulsed at a desired frequency. The device acts to “supercharge” human cells to promote healthy cellular activity, improve blood flow and improve overall health. The device provides a high powered magnetic field that can penetrate 9 to 12 inches of the human body.

The disclosed therapy device provides an alternative treatment for chronic pain and illnesses, including treatment of inflammation and edema associated with such illnesses. The device is known to generate considerable heat in use, and therefore should be utilized up to 5 minutes every hour. The therapy may be administered several times in one day, depending on the severity of the user's condition and the preferences of the user. A timing circuit may be supplied to monitor the operational time and shut down the device if overused within a given time period. Alternatively, a heat sensor may be included to shut down the device in the event of overheating, similar to systems used on laptop computers that monitor heat from an integrated circuit board. To improve heat problems in the coil, a layer of ceramic insulation may be provided to shield the core, or alternatively a copper paste may be supplied to fill the coil core.

Continual use of the magnetic therapy device has been shown to improve health in chronically ill patients and reduce inflammation of tissue and joints. The device provides an easy-to-use therapy means, one that is light weight and portable, and finally one that does not require special tools or appliances to use. The components of the device are commonly available electronic equipment, which provides for an easily producible device that is low cost, to both the manufacturer and the consumer.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. An pulsed electromagnetic therapy device, comprising:
an induction coil embedded in a working end of a handheld stylus, said stylus attached to an electric circuit within a housing;
said electric circuit capable of accepting incoming AC power and transforming it into a pulsing current to said induction coil;
said pulsing controlled by a timing circuit and input from a user via a knob on said housing;
said electric circuit capable of producing a pulsing frequency of between 1 Hz and 25 Hz, and said induction coil capable of creating magnetic fields up to 55,000 mGauss at 2 mm from said coil surface.

2. A method of treating ailments and inflammation using pulsed electromagnetic therapy, comprising the steps of:
placing a handheld stylus against a target region;
pulsing an induction coil at 25 Hz, and with a magnetic field energy of 55,000 mGauss;
pulsing said induction coil for a period of 5 minutes at a time.
